(19) United States
(12) Patent Application Publication
(10) Pub. No.: US 2010/0048867 A1
(43) Pub. Date: Feb. 25, 2010

(54) DEVICE AND PROCESS FOR PURIFYING NUCLEIC ACIDS

(75) Inventor: Markus Mueller, Dormagen (DE)

Correspondence Address: Baker Donelson Bearman, Caldwell & Berkowitz, PC, 555 Eleventh Street, NW, Sixth Floor, Washington, DC, 20004, US

(73) Assignee: QIAGEN GMBH, Hilden (DE)

(21) Appl. No.: 12/524,095

(22) PCT Filed: Jan. 3, 2008
(86) PCT No.: PCT/EP08/50023

§ 371 Date: Aug. 28, 2009

(30) Foreign Application Priority Data
Jan. 31, 2007 (DE) 10 2007 005 655.0

(51) Int. Cl. C12N 15/63; C07H 21/00; C07H 21/02; C07H 21/04; C07K 9/00; B01D 15/08

(52) U.S. Cl. 530/344; 536/25.41; 536/23.1; 210/198.2

(57) ABSTRACT

An apparatus for purifying nucleic acids by negative chromatography is disclosed, which comprises a hollow body with an opening, respectively, at the top and at the bottom end, said hollow body containing a stationary solid phase, characterized in that the stationary phase contains at least 2 different chromatography resins, such as size e.g. exclusion gel filtration materials (SEC materials), as well as a method for purifying nucleic acids and the use of the apparatus.

The present invention relates to an apparatus and a method for purifying nucleic acids as well as to the use of the apparatus.

Various nucleic acids are used in a number of fields, such as recombinant genetic engineering, medical diagnostics, as probes and in many other ways. In these cases, the necessity frequently arises of separating mixtures of nucleic acids of various origins from contaminating substances that interfere with subsequent reactions. Different methods for this are known.

Generally, the nucleic acids are frequently purified and/or concentrated by chromatographic methods in which they are adsorbed to surfaces of, for example, silicon dioxide, silicon dioxide polymers, magnesium silicate or the like, followed by washing and desorption, in each case using suitable solvents. In the process, the undesired contaminants are being removed by washing, and the desired nucleic acids are finally eluted by desorption.

This routine, which in the laboratory jargon is called “bind-wash-elute”, is the result in all purification methods in which the target molecule to be purified (e.g. nucleic acids) is first bound to a carrier material (silica, ion exchanger, hydrophobic interaction chromatography resins, reversed phase chromatography resins, mixed mode chromatography resins, affinity chromatography resins). Although this procedure as a rule yields high-quality preparations of the target molecule, the number of process steps, however, is relatively high.

Obtaining similar degrees of purity with purification methods comprising fewer steps would be desirable.

Particularly suitable for this purpose is size exclusion chromatography (SEC). If an organic solvent is used as the mobile phase, this method is also called gel permeation chromatography (GPC), and gel filtration chromatography in the event an aqueous mobile phase is used. Separation in SEC is based purely on the size (the hydrodynamic volume, to be exact) of the molecules in the sample solution. For similarly shaped molecules, the hydrodynamic radius is proportional to the molecular weight, which is why SEC is also called a mass-based separation method.

A GPC system or SEC system generally comprises a pump, an injection system, separation column(s) and detector(s). The pump draws the eluent (mobile phase), e.g. a suitable elution buffer, and generates a flow through the entire system. The application of the sample onto the separation column, in which the sample is separated because of the different hydrodynamic radius of the constituents, is carried out by the injection system (manually or automatically). Detection is carried out by means of detectors.

The separation columns are filled with the stationary phase, i.e. with small spheres of a porous highly cross-linked material. There are numerous different chromatographic materials for SEC. Every material is characterized by an “exclusion limit” which denotes the suitable upper limit for the molecules that can be separated with the material. The materials used include, for example, polyacrylamide, dextran or agarose or also silica, cross-linked polystyrene or the like. The use of Sephadex®, Sephacryl® or Sepharose® (Pharmacia), Biogel® (Bio-Rad), or Fractogel® (Merck) is widespread.

The separation in SEC is based on the distribution of the molecules between the liquid amongst the spheres of chromatographic material and the liquid in the pores within these spheres. The diameter of the spheres lies in the μm range. If a sample containing molecules of different sizes is eluted, the small molecules enter the pores of the stationary phase and thus remain on the column longer than the larger molecules, which are therefore eluted first. Generally, the columns used have a very small pore size distribution and separate very well in a certain molecular size range. In order to accomplish separation over a larger molecular size range, several separation columns having different pore sizes must be connected in series.

In conventional SEC chromatography of nucleic acids, contaminants having a small molecule size are therefore retained on the column, whereas the desired nucleic acids are eluted together with contaminants such as other undesired nucleic acids, so that further separation steps are subsequently necessary in order to separate these mixtures. This method, in which the desired substances to be separated are first eluted, is also called “negative chromatography”. However, it is problematic, in particular in the case of RNA isolation, for example from PCR reactions, that several nucleic acids of different sizes are eluted together, because RNA is eluted together with large DNA molecules by conventional SEC chromatography, while only smaller moleculesremain on the column and because in that case, RNA has to be separated from the DNAs in a further step.

Ultrafiltration (for particles having a diameter <0.1 μm) and microfiltration (for particles having a diameter of >0.1 μm) are known as further methods for the separation and concentration of high-molecular substances. Both methods are physical membrane separation methods. All the substances contained in liquids, which are larger than the pores of the membrane, are retained by the membrane. The driving force in both methods is the difference in pressure between the inlet and the outlet of the filter surface. The material of the filter surface can include, for example, stainless steel, plastic or a textile tissue. The exclusion limit (cut-off) is usually given as NMWC (Nominal Molecular Weight Cut-off) specified in the unit Dalton (Da).

One DNA ultrafiltration method is disclosed, for example, in DE-A 102 01 487.

US 2004/0002081 discloses a method for isolating and purifying a polynucleotide on an industrial scale, which utilizes a chromatographic method in which at least two different chromatographic steps, selected from hydrophobic interaction chromatography, polar interaction chromatography and anion exchange chromatography, are carried out, wherein the chromatographic carrier in at least one step is a porous monolithic bed.

EP-A 1 719 16 discloses a material for separating and purifying DNA which is an integral porous monolithic body of glass or silica having pores going through from one end to the other, said pores corresponding to nucleic acid sizes of 35 bp to 100 kbp, so that the corresponding nucleic acids are retained in these pores.

WO 01/94574 discloses a material for purifying DNA sequencing reaction products, that is, for separating the DNA sequencing fragments from sequencing templates, enzymes, salts and nucleotides, wherein a filtration medium is combined with a molecular cutoff filter in a single chromatography column. For example, an SEC material such as Sephadex® G-50 or Sephacryl® is combined with a Whatman-Polyfiltronics® ultrafiltration membrane.

EP-A-1 589 337 discloses a packing material for liquid chromatography comprising particle mixtures of at least two components for separating small molecules, proteins or nucleic acids. The particles can comprise any inorganic and/or inorganic-organic hybrid materials which are suitable for chromatographic application, such as silica particles. The two components each have a particle size distribution and a mean particle diameter, wherein the difference between any two mean diameters is less than or equal to 40% of the larger mean value.

WO 2004/011142 discloses a device for purifying a sample containing, for example, nucleic acids, wherein an SEC material and an ion exchange material (IEC material) are combined in said device. In particular, so-called “size-exclusion ion-exchange” (SEIE) particles are being disclosed, wherein, for example, a core of an ion exchange material capable of size exclusion is encapsulated by a shell of an SEC material. It is also possible to use several such SEIE materials in a single chromatography column.

Generally, the known chromatography methods on an mg-scale (relative to the substance to be separated) are carried out on conventional separation columns, with the dimensions of the column and the quantity of the stationary phase being suitably adapted to the quantity of the substance to be purified. For elution, the solvent is introduced into the upper end of the column under pressure (generated by pumps).

Substantially smaller chromatography devices for purifying smaller quantities of less than 10 mg of substance to be purified are also known. For example, the stationary phase is introduced into the wells of microwell plates, the mixture to be separated is filled into the wells, eluent is introduced into the wells and then eluted by applying a vacuum, or the stationary phase is introduced, on a frit, into an Eppendorff tube open at the bottom, the substance to be separated and the eluent are applied, and then, the tube is inserted into a centrifuging tube and elution is effected by centrifuging, so that the eluate collects in the centrifuging tube and can be withdrawn therefrom and processed further. In this way, chromatography is carried out, for example during the processing of PCR reaction mixtures, by centrifuging with DyeEx® (Qiagen GmbH), in order to separate color markers and primers from the PCR products.

Chromatography devices in microfluidic chips are also known.

In all these small-scale methods, however, only the use of a single SEC material per chromatography step is known in the state of the art, so that several chromatography steps possibly have to be carried out one after the other.

One problem of the known devices and methods for purifying DNA therefore lies in the fact that several columns always have to be connected in series in order to combine several selectivities. Therefore, adapting the columns to certain desired selectivities always consumed a lot of time and material.

Therefore, a chromatography apparatus for nucleic acids, or the combination of chromatography and membrane methods, was desired with which the selectivity or the retention time and the exclusion properties (cut- off) can be set easily. Moreover, there was a demand for a single-step method in which the desired nucleic acids of a certain size can be separated in a single step both from smaller contaminants as well as from larger nucleic acids.

Therefore, it is the object of the invention to provide such an apparatus.

The object is achieved by the apparatus for negative chromatography according to claim 1. Preferred embodiments are defined in the claims 2 through 6. Moreover, a method for purifying DNA using the apparatus according to the invention as well as the use of the apparatus are being claimed.

According to the invention, SEC materials of different selectivities are combined for the first time and used as a mixture or in layers in a single apparatus. So far, it was always necessary to connect several chromatography devices in series that respectively contained different SEC media. Due to the apparatus according to the invention, it is therefore possible to save a considerable amount of time. A selectivity that is adjustable at will is possible in particular due to the combination with micro or ultra filtration membranes, which is claimed as a preferred embodiment. So far, such membranes were not combined with SEC materials in order to further adjust selectivity in this manner.

A great variety of nucleic acids, such as genomic DNA, plasmid DNA, various RNA's, PCR fragments, oligos or restriction fragments, can be purified and/or concentrated using the apparatus according to the invention. The apparatus is particularly suitable for separating RNAs from mixtures containing larger DNAs. Generally, however, this principle of separation can lead to a separation of the protein fraction from the RNA fraction and gDNA fraction of a sample and be available for further analyses, so that a parallel downstream analysis of the genome, the transciptome and the proteome is possible. Moreover, the separation principle described can be used for other applications such as plasmid purification etc. Generally, the samples are present in the form of a solution, with saline, clarified cell lysates usually being used as solvents. The solution can be a product solution obtained directly from a reaction, or a sample solution obtained after any type of processing. Prior to application onto the apparatus according to the invention, the sample can be prepared, for example, by filtering or centrifuging (e.g. clarification of alkaline bacterial lysates.

All known SEC materials can be used as SEC materials, e.g. Superdex®, Sephacryl® or Superose® (Pharmacia), Biogel® and Biosil® (Bio-Rad), and Fractogel EMD BioSEC® (Merck). SEC materials with exclusion ranges (cut-off) of 100 to 500,000 Da or more are available. The materials are combined in a suitable manner such that the desired size selection is achieved as desired in one step, custom-tailored to the target molecule. By means of a suitable selection of the SEC combination, undesired contaminants such as salts, metabolites, proteins, undesired nucleic acids, fats etc. can be retained on the column, whereas the desired nucleic acids, but also protein factors, are eluted and can thus be isolated.

Moreover, it is conceivable to add further selectivities within such a “composite” column, such as anion exchangers and HIC functionalities or resins. For example, the combination of an anion exchanger resin, layered on top of a desalting gel filtration material within a centrifuging or vacuum tube would be suitable for the step-by-step elution of desalted protein, RNA and gDNA fractions.

The outer shape of the apparatus is not limited in any particular way. Any conventional shape used for chromatography can be used. Usually, round columns of glass or plastics that comprise openings at the top and the bottom end and whose diameter is small compared to the length, are used. Preferably, the apparatus contains a frit of glass or another suitable material at the bottom end. Furthermore, it preferably comprises means for applying a vacuum or for generating overpressure; these are also generally known.

The size of the apparatus is also not limited. It can be designed for separation processes from the nanogram range up to quantities of several grams to kilograms. Therefore, work on laboratory scale (ng to μg scale) up to the preparatory scale (g to kg scale) can be done with the apparatus according to the invention.

According to the invention, the apparatus may also be in the form of a microwell plate, into whose wells the SEC materials are introduced in a largely homogeneously mixed form or in layers, or even in the form of tubes open at the bottom, e.g. Eppendorff tubes, into which the SEC media are introduced in a largely homogeneously mixed form or in layers. In this case, the tubes expediently comprise a frit at the bottom end, so that the SEC medium is retained in the tubes. The apparatus can also exist as a cavity in a microfluidic chip.

According to the invention, the SEC materials can either be mixed prior to filling the apparatus and then filled into the apparatus as a largely homogeneous mixture, or they are filled in in layers, with the layer thicknesses being suitably selected depending on the capacity of the selected material for the contaminant to be removed, in the stochiometric ratio to the presence of such a contaminant in the sample. The layer thicknesses of the individual layers can be approximately the same or also different. Surprisingly, an increase of the separation performance as compared with the corresponding mixed bed mode is achieved by filling in layers.

With regard to the connections, the apparatus is configured such that they are operated in the centrifuging, vacuum, pressure or gravity mode. This is the conventional configuration of a chromatography apparatus known in the prior art. It is particularly preferred to use the apparatus for separations on a small scale (up to 10 mg of substance to be separated) and to then effect elution by vacuum, pressure or centrifuging.

Moreover, the apparatus according to the invention particularly preferably comprises one or more micro or ultra filtration membranes which are arranged underneath and/or above the SEC media. The membranes can also be disposed within the SEC materials, that is, either the mixture or individual layers can be disposed both above as well as underneath the membrane. These membranes are selected from those that are commonly commercially available, e.g. micro filtration membranes with cut-off ranges of between 0.2 μm to 10 μm, for coarse clarification e.g. Miracloth® (Calbiochem) and ultra filtration membranes between 50 and 1000 kDa.

According to the invention, the SEC materials and the micro or ultra filtration membranes are suitably selected in accordance with the size of the nucleic acids to be isolated. Large nucleic acids can be purified either by reverse purification with SEC media with large exclusion sizes, or they can be retained by micro or ultra filtration with membranes having a suitable cut-off. Smaller nucleic acids can be retained on the column by the combination of various SEC materials with suitable cut-offs. Mixing different SEC materials with different size selectivities thus makes the retention of undesired contaminants possible, while the desired nucleic acids are being eluted. The combination of several SEC materials with micro and/or ultra filtration membranes can further increase the size selectivity in a single purification step. The same applies for the addition of further separation principles within the same column body. According to the invention, it is thus possible for the first time to separate, on the one hand, for example, RNAs in a single purification step from small contaminants such as templates, primers, fats, salts, metabolites, proteins, which are retained on the column by the suitably selected SEC materials, and at the same time to separate the undesired larger nucleic acids such as genomic DNA also in the same step, because they are retained and separated by the UF/MF membrane. In a variant of this use, the genomic DNA is first precipitated by selectively precipitating agents prior to the application to such a composite column, which then makes a separation by larger-pore filter materials from the micro and deep-bed filtration range possible.

Suitable conventional elution or running buffers or solvents such as water or lightly buffered solution such as tris, MOPS and citrate buffer are used as an elution medium.

The eluate is collected in fractions of any size.

Detection is done by conventional detectors, e.g. with ultraviolet light (UV detector), or with functional assays such as PCR reactions.

The fractions containing the desired nucleic acids are further analyzed either directly in downstream reactions or concentrated for further analyses by state-of-the-art methods (precipitation/resuspension, ultrafiltration, binding to another column and concentrated elution).

### EXAMPLES

A 2.0 ml plastics reaction vessel with an outlet at the bottom end is first filled with 500 μl Sephadex G 50 and then with 500 μl Sepahcryl S 500. Both materials are retained in the reaction vessel by the frit of suitable, commercially available material located in front of the opening. The vessel is centrifuged after the application of 1 ml clarified, plasmid-containing bacterial lysate. Purified and desalinated plasmid DNA is located in the passage.

By placing an ultrafiltration membrane, for example with a cut-off of 500 kDa, above the frit or above the resin bed, contaminated gDNA components can be retained on the column and prevented from co-eluting with the plasmid DNA.

1. An apparatus for purifying nucleic acids by negative chromatography comprising a hollow body with an opening, respectively, at a top and at a bottom end thereof, said hollow body comprising a stationary solid phase, wherein the stationary phase comprises at least two different chromatography resins.

2. An apparatus according to claim 1, further comprising at least one ultrafiltration and/or microfiltration membrane.

3. An apparatus according to claim 1, further comprising at least one chromatography resin of additional selectivits.

4. An apparatus according to claim 1, wherein the at least different resins comprise at least one SEC material are present in a largely homogeneous mixture.

5. An apparatus according to claim 4, comprising SEC material arranged in layers.

6. An apparatus according to claim 1, further comprising at least one.

7. An apparatus according to claim 1, further comprising a connecting means for applying a vacuum or overpressure, for centrifuging and/or for detection.

8. An apparatus according to claim 1 in the form of a microwell plate and/or in the form of an Eppendorff tube.

9. A method for purifying nucleic acids, comprising using an apparatus according to claim 1.

10. A method for purifying nucleic acids comprising using an apparatus of claim 4.

11. A method or use according to claim 9, wherein the nucleic acids are selected from the group consiting of genomic DNA, plasmid DNA, RNA, PCR fragments and oligos.

12. An apparatus of claim 1, wherein the at least two resins comprise a size exclusion gel filtration material.

13. An apparatus according to claim 2, wherein the at least different resins comprise at least one SEC material present in a largely homogeneous mixture.

14. An apparatus according to claim 3, wherein the at least different resins comprise at least one SEC material present in a largely homogeneous mixture.

15. An apparatus according to claim 2, further comprising at least one frit.

16. An apparatus according to claim 3, further comprising at least one frit.

17. An apparatus according to claim 2 in the form of a microwell plate and/or in the form of an Eppendorff tube.

18. An apparatus according to claim 3 in the form of a microwell plate and/or in the form of an Eppendorff tube.

19. A method for purifying nucleic acids, comprising using an apparatus according to claim 2.

20. A method for purifying nucleic acids, comprising using an apparatus according to claim 3.
